(19) United States
(12) Patent Application Publication
(10) Pub. No.: US 2013/0072924 A1
(43) Pub. Date: Mar. 21, 2013

(54) ABLATION ANTENNA

(71) Applicants: Robert H. Burgener, Park City, UT (US); Todd H. Turnlund, Park City, UT (US); Chet M. Crump, South Jordan, UT (US); Kent Moore, Bountiful, UT (US)

(72) Inventors: Robert H. Burgener, Park City, UT (US); Todd H. Turnlund, Park City, UT (US); Chet M. Crump, South Jordan, UT (US); Kent Moore, Bountiful, UT (US)

(73) Assignee: BSD MEDICAL CORPORATION, Salt Lake City, UT (US)

(21) Appl. No.: 13/615,017

(22) Filed: Sep. 13, 2012

Related U.S. Application Data
(60) Provisional application No. 61/536,680, filed on Sep. 20, 2011.

(51) Int. Cl. A61B 18/18; H01P 11/00

(52) U.S. Cl. 606/33; 29/600

(57) ABSTRACT

A radio frequency ablation antenna is disclosed. The micro-strip ablation antenna has a dielectric member having a substantially tubular shape. A first conductor is disposed within the dielectric member, and a second conductor is disposed on an outer surface of the dielectric member. The first conductor is configured to be electrically connected to a radio frequency source or ground, and the second conductor is configured to be electrically connected to the other of the radio frequency source or the ground.

### RELATED APPLICATION

This application claims the benefit of U.S. Provisional Application No. 61/536,680 filed Sep. 20, 2011, entitled MICROWAVE ABLATION ANTENNA, which is incorporated herein by reference.

### BACKGROUND

Radio frequency ablation (RFA) is a medical procedure where in vivo tissue is ablated using high frequency alternating current to treat a medical disorder. RFA is commonly performed to treat tumors in body organs. During RFA, a needle-like RFA probe is placed inside the tumor. Radio frequency waves emitted from the probe heat surrounding tumor tissue, destroying the target tissues, such as a cancerous tumor, nerve, or other target structure. Cancer cells, in particular, can break down and die at elevated temperatures caused by radio frequency ablation procedures. Some RFA procedures, such as microwave ablation (MWA) procedures, use temperatures up to or exceeding 300 degrees Celsius. Despite recent advances in RFA antenna designs, improvements are desirable.

### SUMMARY

In some aspects of the present invention, a radio frequency ablation (RFA) device includes a dielectric member, a first conductor disposed within the dielectric member, and a second conductor disposed on an outer surface of the dielectric member. The dielectric member may take any shape and configuration, including multiple conjoined shapes incorporated into one device. In one aspect, the dielectric member has a substantially tubular shape. The first conductor is configured to be electrically connected to a radio frequency source or ground, and the second conductor is configured to be electrically connected to the other of the radio frequency source or the ground.

In another aspect, a method of manufacturing a RFA antenna includes at least the following steps: providing an inner conductor; depositing a layer of dielectric material on the exterior of the center conductor, the layer of dielectric material forming a tubular shape; and depositing an outer conductor on an outer surface of the layer of dielectric material.

In yet another aspect, a microwave ablation (MWA) device includes a probe member and a microstrip antenna element disposed within the probe member. The microstrip antenna element comprises a dielectric substrate having a dielectric constant of between about 4 and about 30. The dielectric substrate has a first substantially flat surface and a second substantially flat surface. The second surface is opposite the first surface. The microstrip antenna element also comprises a first conductor a first conductor disposed on the first surface of the dielectric substrate and a second conductor disposed on a second surface of the dielectric substrate. The second conductor is a microstrip trace. The first conductor is configured to be electrically connected to one of a radio frequency source or ground, and the second conductor is configured to be electrically connected to the other of the radio frequency source or the ground.

In yet another aspect, a RFA device comprises a RFA ablation probe member and a helical dipole antenna element disposed within the probe member. The helical dipole antenna element comprises a first conductor and a second conductor. The first conductor and the second conductor each extend in a substantially parallel direction along a longitudinal axis of the helical dipole antenna to a center point of the helical dipole antenna. The first conductor is wound helically about the longitudinal axis in a distal direction from the center point, and the second conductor is wound helically about the longitudinal axis in a proximal direction from the center point.

### BRIEF DESCRIPTION OF THE DRAWINGS

In order that the above-recited and other features and advantages of the disclosure may be readily understood, a more particular description is provided below with reference to the appended drawings. These drawings depict only exemplary embodiments of radio frequency devices according to the present disclosure and are not therefore to be considered to limit the scope of the disclosure.

FIG. 1 is a partial cross section view of a probe member entering a target tissue within a patient in accordance with some embodiments of the invention.

FIG. 2 is a cross-section view of a probe member in accordance with some embodiments of the invention.

FIG. 3 is a perspective view of an antenna element in accordance with some embodiments of the invention.

FIG. 4 is a cross section view of an antenna element having a helical-shaped outer conductor in accordance with some embodiments of the invention.

FIG. 5 is a cross section view of another antenna element having a helical-shaped outer conductor wherein the antenna element is disposed around the end of a coaxial cable in accordance with some embodiments of the invention.

FIG. 6 is a cross section view of another antenna element having a helical-shaped outer conductor in accordance with some embodiments of the invention.

FIG. 7 is a cross section view of an antenna element having two helical-shaped conductors in accordance with some embodiments of the invention.

FIG. 8 is a cross section view of an antenna element having three helical-shaped conductors and which is configured to operate as a two-phase antenna element in accordance with some embodiments of the invention.

FIG. 9 is a partial cross section view of an antenna element and an adjustable sleeve coupled to the radio frequency feed line in accordance with some embodiments of the invention.

FIG. 10 is a cross-section view of an antenna element having an outer conductor coupled to a plurality of conductive particles in accordance with some embodiments of the invention.

FIG. 11 is a cross-section view of an antenna element having an inner conductor coupled to a plurality of conductive particles within a dielectric member in accordance with some embodiments of the invention.

FIG. 12 is a cross-section view of an antenna element having an inner conductor coupled to a plurality of conductive wires within a dielectric member in accordance with some embodiments of the invention.

FIG. 13 is a perspective view of a conductor having a fractal pattern and which is disposed on the exterior of a dielectric member in accordance with some embodiments of the invention.

FIG. 14 is a perspective view of a conductor disposed on only a portion of the exterior of a dielectric member in accordance with some embodiments of the invention.

FIG. 15 is a perspective view of an antenna element having a planar conductor in accordance with some embodiments of the invention

FIG. 16 is a perspective view of an antenna element having a set of planar conductors in accordance with some embodiments of the invention

FIG. 17 is a perspective view of a helical dipole antenna element in accordance with some embodiments of the invention.

### DETAILED DESCRIPTION

This specification describes exemplary embodiments and applications of the invention. The invention, however, is not limited to these exemplary embodiments and applications or to the manner in which the exemplary embodiments and applications operate or are described herein. Moreover, the Figures may show simplified or partial views, and the dimensions of elements in the Figures may be exaggerated or otherwise not in proportion for clarity. In addition, the singular forms “a,” “an,” and “the” include plural referents unless the context clearly dictates otherwise. Thus, for example, reference to a terminal includes reference to one or more terminals. In addition, where reference is made to a list of elements (e.g., elements a, b, c), such reference is intended to include any one of the listed elements by itself, any combination of less than all of the listed elements, and/or a combination of all of the listed elements.

Numerical data may be expressed or presented herein in a range format. It is to be understood that such a range format is used merely for convenience and brevity and thus should be interpreted flexibly to include not only the numerical values explicitly recited as the limits of the range, but also as including all the individual numerical values or sub-ranges encompassed within that range as if each numerical value and sub-range is explicitly recited. As an illustration, a numerical range of “about 1 to 5” should be interpreted to include not only the explicitly recited values of about 1 to 5, but also include individual values and sub-ranges within the indicated range. Thus, included in this numerical range are individual values such as 2, 3, and 4 and sub-ranges such as 1-3, 2-4, and 3-5, etc. This same principle applies to ranges reciting only one numerical value and should apply regardless of the breadth of the range or the characteristics being described.

By the term “substantially” is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

The term “proximal” is used to denote a portion of a device which, during normal use, is nearest the user wielding the device and furthest from the patient. The term “distal” is used to denote a portion of a device which, during normal use, is farthest away from the user and closest to the patient.

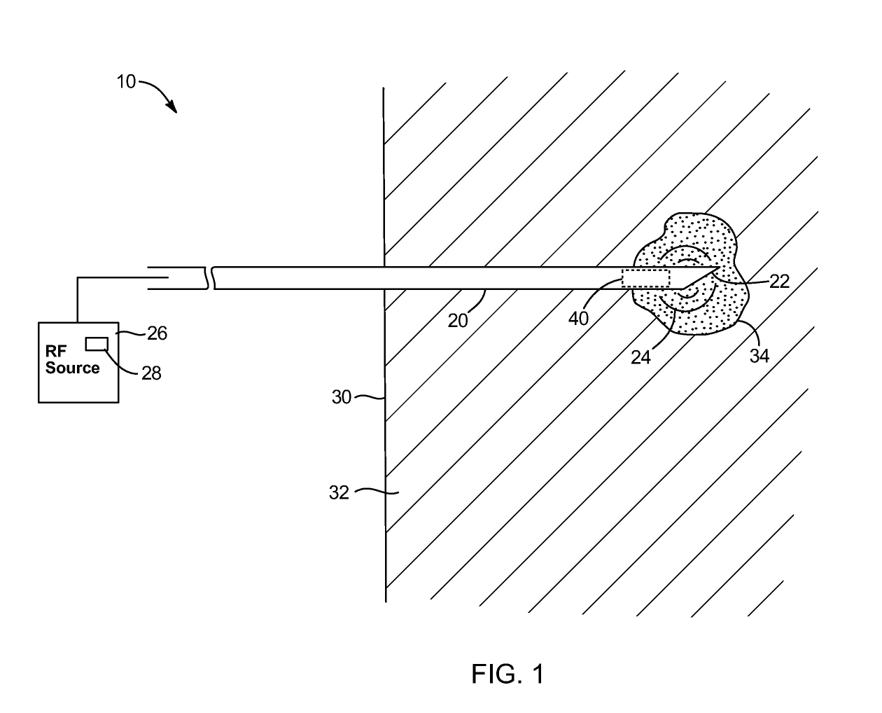

FIG. 1 illustrates a radio frequency ablation (RFA) device 10 that can be used in RFA procedures. The RFA device 10 can include a probe member 20 (or ablator) that includes an elongated shaft and has a distal end 22 that forms a beveled edge, pointed tip, or other like cutting member. This distal end 22 can facilitate penetration of the ablation needle 20 through the skin 30, tissue 32, and target tissue 34 of a patient. Moreover, a distal portion of the probe member 20 can include an antenna element 40. The shaft of the probe member 20 can have various lengths, such as a length of between about 1 inch to about 12 inches or more than 12 inches. The gauge of the shaft can range between 8 to 24, including, but not limited to, a 12, 14, 16, 17, or 18 gauge shaft. An example of a probe member 20 is the SynchroWave Antenna from BSD Medical Corporation of Salt Lake City, Utah.

The RFA device 10 can also include a radio frequency power source 26 that is connected to the probe member 20. The radio frequency power source 26 can deliver radio frequency energy to the antenna element 40 of the probe member 20. Moreover, the radio frequency power source 26 can include a controller 28. The controller 28 can control the power, frequency, and/or phase of the energy delivered to the antenna element 40 of the probe member 20. For example, when two or more probe members 20 are connected to the radio frequency power source 26 the controller 28 can control the power, frequency, and/or phase of energy delivered to two or more probe members. In another example, the controller 28 can control the power frequency and/or phase of energy delivered to two separate conductors of a single antenna element 40, such as the antenna element 40 shown in FIG. 8. In some embodiments, the controller 28 can also be configured to automatically adjust the power, frequency, and/or phase of the energy delivered to an antenna element 40 in order to automatically tune or impedance match the antenna element 40 to the target structure 34.

The RFA device 10 can be configured to transmit energy having one or more frequencies or a variable frequency. For example, in some embodiments, the radio frequency power source is a microwave source configured to provide microwave energy to the antenna element 40. Such energy can have a frequency within the range of about 880 to 960 MHz, including specifically, for example, 915 MHZ. When microwave energy is delivered to the antenna element 40, tissue surrounding the antenna element 40 can be ablated (heated, burned or cooked) with heat generated by the antenna element 40. In other embodiments, energy delivered by the radio frequency power source 26 can have a frequency within the range of about 400 MHz and about 4 GHz.

Additionally, the radio frequency power source 26 can be configured to transmit various levels of energy to the antenna element 40. In some embodiments, the radio frequency power source 26 can transmit up to about 300 W of power to the antenna element 40. In other embodiments, the radio frequency power source 26 can transmit between 0 W to 300 W of power to the antenna element 40, including specifically transmitting up to 40 W, up to 60 W, up to 120 W, up to 180 W, or up to 240 W of power to the antenna element 40.

In some embodiments, the controller 28 can be configured to ramp up the power delivered to an antenna element 40 slowly during the initial phases of an ablation procedure. Such configurations can incrementally or exponentially or otherwise ramp up power from zero to a maximum power output over a predetermined time. For instance, the controller 28 can be configured to ramp up power delivered to the antenna element 40 from 0 W to 60 W over a two-minute period. Stepping or ramping up the power can assist to retain water or water vapor in the ablated area and thus increase the size of the ablated area over time. In contrast, rapidly applying high power can carbonize the ablated area, which makes it more difficult to increase the size to the ablation region. An example of a radio frequency source having a controller is the MicroThermX® Microwave Ablation System from BSD Medical Corporation of Salt Lake City, Utah.

As shown in FIG. 1, the RFA device 10 can be used in ablation procedures. Such procedures involve ablating in vivo tissue using high frequency alternating current. During RFA, the probe member 20 is inserted through the skin 30 and tissue 32 of a patient, and is then directed toward a target structure 34, such as a tumor, cell(s), or nerve(s). The probe member 20 can be inserted into the target structure 34, as shown, or placed beside the target structure 34. Radio frequency energy 24 emitted from the probe member 20 can then heat the target structure 34, which may be burned and/or killed. When the target structure 34 is exposed to the transmitted radio frequency energy for an adequate amount of time, the target structure 34 can be ablated. Cancer cells, in particular, can break down and die at elevated temperatures caused by radio frequency ablation procedures. Some RFA procedures, such as microwave ablation (MWA) procedures, use temperatures up to or exceeding 100, 200, 300, and 350 degrees Celsius.

Generally, the shape and size of ablation pattern produced by the antenna element 40 roughly corresponds to the shape and intensity of the radio frequency transmission patterns of the waves 24 emitted from the antenna element 40. Thus, a substantially spherical transmission pattern can produce a roughly spherical ablation pattern. Accordingly, the RFA device 10 can be configured to produce ablation regions that are substantially the same size as the targeted structure 34 so that the appropriate amount of target tissue is ablated, without ablating healthy surrounding tissues. For example, since many tumors are approximately spherical, the RFA device 10 can be configured to produce a generally spherical ablation region. Such a spherical ablation region can be produced using one of the antenna elements 40 shown in the following Figures.

Additionally, the RFA device 10 can be configured to produce ablation regions that are directional and manipulable (or shapeable) so that they can be shaped to be the same size as a target structure 35 or so that they can be directed toward a target structure near a probe member 20. Such directionality can be produced, in some instances, by varying the phase between transmitted radio frequency energy transmitted through multiple conductors of the antenna element 40, as shown in FIG. 8 and described with reference to that Figure.

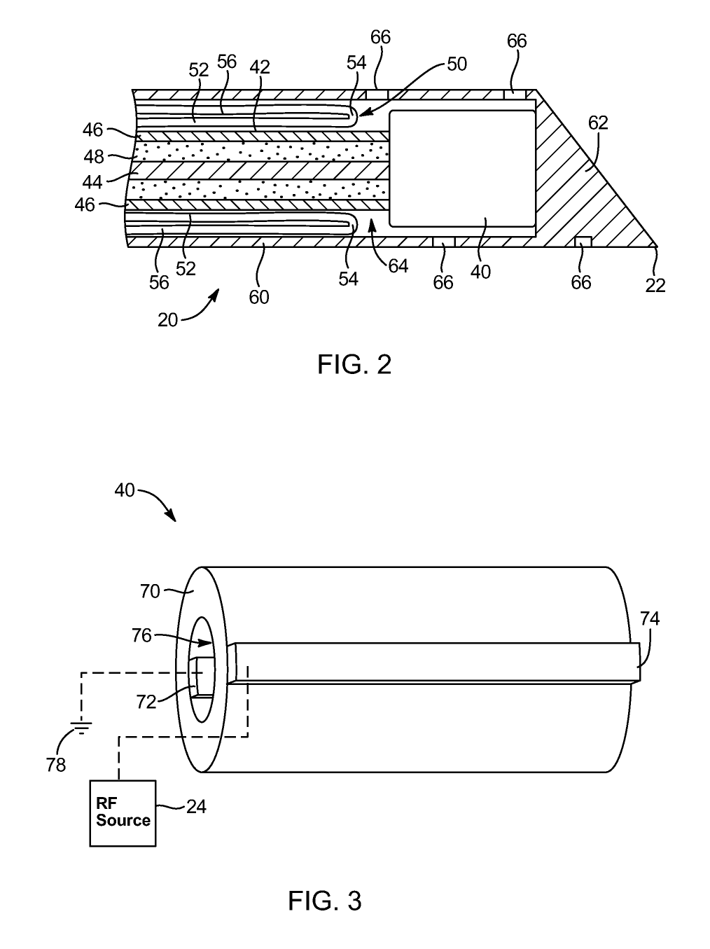

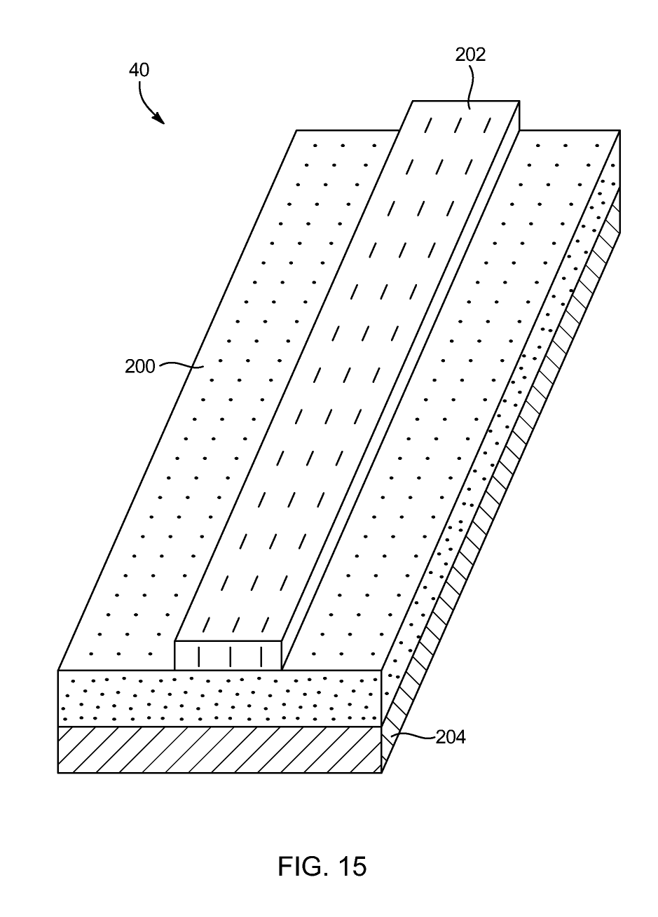

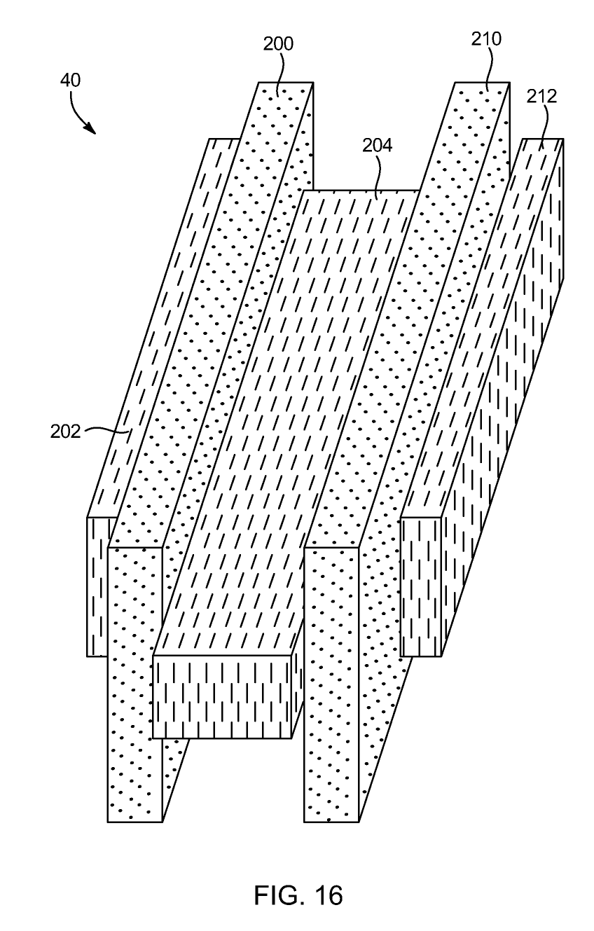

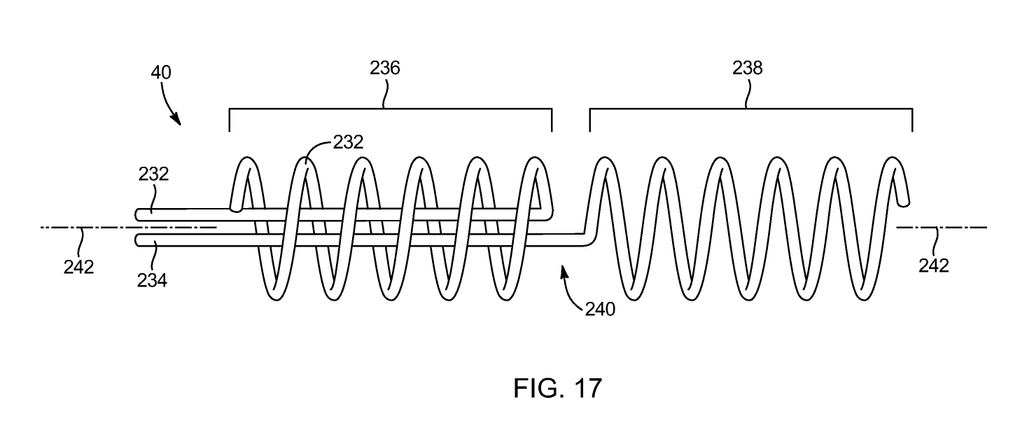

FIG. 2 illustrates a cross-sectional view of a distal portion of a probe member 20. The probe member 20 can include a shaft 60 that has an inner cavity 64 and a tip 62. An antenna element 40, coaxial cable 42, and/or cooling tube 50 can be disposed within the inner cavity 64. Various types of antenna elements 40 can be incorporated into the probe member 20. For example, the antenna element 40 can be a generally tubular or cylindrical microstrip-type antenna element (e.g., antenna element 40 of FIGS. 3-14), a planar microstrip-type antenna element (e.g., antenna element 40 of FIGS. 15-16), a helical dipole antenna element (e.g., antenna element 40 of FIG. 17), or others types of antenna elements.

The antenna element 40 can be connected to a coaxial cable 42, which can be used to electrically couple the antenna element 40 the radio frequency power source 24 (shown in FIG. 1) and ground or a common line. The coaxial cable 42 can include an inner conductor 44 and an outer conductor 46 separated by a dielectric material 48. Moreover, in some embodiments, the coaxial cable 42 can include more than one inner conductor 44. For example, the coaxial cable 42 can include two, three, four, or more inner conductors 44. FIG. 8 illustrates an example of a coaxial cable 42 that includes two inner conductors 44. In other embodiments, three, four, or more than four inner conductors 44 are disposed within the coaxial cable 42. When multiple inner conductors 44 are used, separate signals or signals with different phases, frequencies, etc. can be transmitted down each inner conductor 44. In some embodiments, the coaxial cable 42 can have a gauge of between about 10 to about 20.

In some embodiments, the inner conductor 44 can be a radio frequency feed line and the outer conductor 46 can be grounded. In other instances, inner conductor 44 can be grounded and the outer conductor 46 can be connected to the feed line. However, for the purpose of this application it will be assumed that the inner conductor 44 is a radio frequency feed line that is connected to the radio frequency power source 24 and that the outer conductor 46 is grounded.

Referring still to FIG. 2, the probe member 20 can include a cooling system that cools at least a portion of the shaft 60 to prevent damage to the patient's skin and other tissues in contact with the shaft 60. The cooling system can include a cooling fluid, such as water, saline, or another fluid, that circulates through one or more cooling tubes 50, cooling channels, or cooling jackets disposed within the inner cavity 64. Moreover, a thermal electric (TE) cooler can be incorporated into the cooling system to provide additional cooling to the main cooling reservoir such as an IV fluid bag containing fluid that is pumped through the cooling system. Additionally, the cooling system can include a pump (not shown) for circulating fluid through the cooling tubes 50.

In an example, as shown, the cooling system includes at least two cooling tubes 50, each having an inflow portion 52 and an outflow portion 56 joined by a bend 54. In operation, fluid flows down the inflow portion 52, around the bend 54, and back through the outflow portion 56. In another example, the cooling system can include a baffle return system, a heat transfer conduction pipe, or heat pipe (not shown). As shown, the cooling system can terminate close to the proximal end of the antenna element 40. In other configurations, the cooling system can pass through or around the antenna element 40.

As further shown in FIG. 2, in some embodiments, the probe member 20 includes one or more thermisters or sensors (collectively “sensors”) 66 disposed thereon. These sensors 66 can be coupled to the inside or outside of the shaft 60 and/or embedded therein. These sensors 66 can also be disposed internally or externally at a distal portion of the probe member 20, near or adjacent to the antenna element 40. Additionally, one or more sensors 66 can be displaced along the shaft 60 so as to provide a reference measurement to the controller 28.

The sensors 66 can be electronically coupled to the controller 28 to provide various measurements used in controlling the operation of the RFA device 10. For example, the sensors 66 can be configured to detect changes in tissue, tissue impedance, tissue consistency, temperature, moisture levels, and the like near the sensors. Example sensors 66 include a sputtered resistive film junction of seabeck, a P-N junction, a thermocouple, a temperature sensor, or the like. In some embodiments, the sensors 66 can be referenced frequency dependent and/or tuned to a specific frequency. For example, a capacitor can be disposed between each sensor 66 and the controller 28.

In some embodiments, one or more of the sensors 66 can be configured to sense the temperature of tissues or other structure in proximity to the sensor. Temperature feedback can be used to control the power level of the energy supplied to the antenna element 40. Using this temperature feedback, the controller 28 can control oblation temperatures to prevent the early boiling of water within the target structure 34. This can prevent carbonization of tissues surrounding the antenna element 40 and thus decrease oblation time and increased power efficiency.

Additionally or alternatively, the sensors 66 can be configured to sense the dielectric properties of tissues or other structures in contact or in proximity to the sensor 66. Thus configured, the sensors 66 can distinguish between different types of tissues including healthy tissues and diseased tissues.

Referring still to FIG. 2, the antenna element 40 can be enclosed or sealed by a sealing member (not shown). The sealing member can protect the antenna element 40 from exposure to the patient's tissues 32 and/or fluids and prevent electrical interference therewith. For example, in some embodiments, sealing member is a layer of epoxy, glass, or other such materials. In other embodiments, the sealing member is a ceramic or plastic tube, etc. Other types of sealing members can be used to protect the antenna element, particularly members that can withstand the heat of the ablation procedure. In some embodiments, the sealing member can be disposed in close contact with the outer surface of the antenna element 40. In other embodiments, there can be a space between the sealing member and the outer surface of the antenna element 40.

Because the ablation process heats tissue based, at least in part, upon the moisture content in the tissue, in some instances, it can be useful to minimize the moisture loss that can result during ablation. Accordingly, in some embodiments, a barrier or separator is placed between the dielectric and the target tissue. A non-limiting example of such a barrier includes a silicon inflation balloon coupled to or otherwise associated with the probe member 20. The balloon can be inflated using gas pressure. The inflated balloon can compress the tissue and retain moisture therein. Another non-limiting example of a barrier or separator includes one or more expandable stints.

In some configurations, the probe member 20 and other components of the RFA device 10 can be configured to be sterilized multiple times. Accordingly, the probe member 20 can include a protective cover, coating, or other such protection that is configured to withstand the ablation process and the sterilization process. Such protection can be made of a medical grade material.

FIG. 3 illustrates an example of an antenna element 40 in accordance with some embodiments of the invention. In some embodiments, this antenna element 40 can replace the antenna element 40 shown in FIG. 1 or 2.

As shown, the antenna element 40 can include a dielectric member 70 that has a substantially tubular or cylindrical shape. For example, the dielectric member 70 can form a lengthy or blunt tube or cylinder. The tubular-shaped dielectric member 70 can include an inner void that extends through the entire length of the tube. This void can be filled with another structure. Moreover, the tubular-shaped dielectric member 70 can be formed as a layer or coating on another object. Moreover still, the tubular-shaped dielectric member 70 can be formed as a sleeve or separate component. In tube configurations, the tube can have a variety of inner and outer shapes, including, but not limited to a perfect or imperfect square, circle, oval, ellipses, triangle, other polygon, or other suitable shape.

A first conductor, an inner conductor 72, can be disposed within and/or coupled to the dielectric member 70. The inner conductor 72 can be disposed on an inner surface of the dielectric member 70 including on an interior surface of an inner lumen 76 of the dielectric member 70. Additionally, a second conductor, an outer conductor 74, can be disposed on and/or coupled to an outer surface of the dielectric member 70. In some embodiments, the inner conductor 72 is electrically connected to the radio frequency power source 24 and the outer conductor 74 is electrically connected to ground 78. In other embodiments, as shown, the inner conductor 72 is electrically connected to ground 78 and the outer conductor 74 is electrically connected to the radio frequency power source 24.

The use of a tubular or cylindrical dielectric member 70 can enable the inner conductor 72 and the outer conductor 74 to have various configurations that are disposed around the entire inner or outer surfaces, respectively, or on only a portion of the inner or outer surfaces such as on one side, one quadrant, two quadrants, three quadrants, and/or a potion of a quadrant. This versatility can enable the antenna element 40 to be configured to provide a uniform radiation pattern about the entire antenna element 40 or to provide a customized or directional radiation pattern. The resulting radiation patterns can result from the configuration of the inner conductor 72 and the configuration of the outer conductor 74 along with the connection of the radio frequency power source 24 to either the inner conductor 72 or the outer conductor 74. Additionally, the use of a tubular or cylindrical dielectric member 70 can enable the inner conductor 72 and/or the outer conductor 74 to be disposed on the dielectric member 70 in a nonlinear pattern so that the dielectric member 70 can have a shorter overall length. As such, the antenna element 40 can operate more like to a point source and thus is capable of producing a relatively spherical ablation pattern.

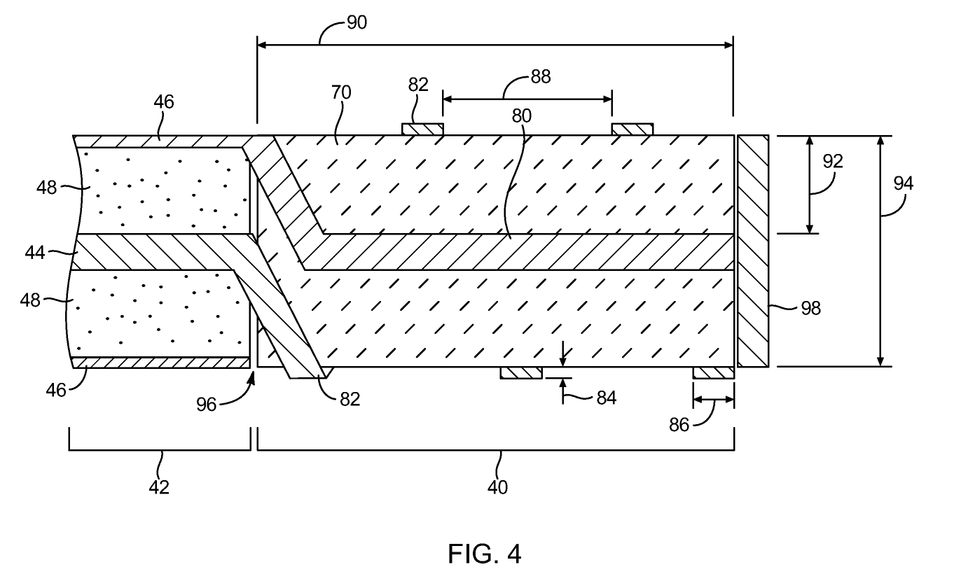

Both the inner conductor 72 and the outer conductor 74 can have a variety of shapes, sizes, and configurations. For example, as shown, the inner conductor 72 can be a relatively straight or linear strip of material that extends between a distal end and a proximal end of the dielectric member 70. Alternatively, the inner conductor 72 can be a strip of material having a nonlinear pattern, such as a zigzag pattern, helical pattern, fractal pattern, back-and-forth pattern, set of radial rings, set of radial bands, or other suitable pattern. In another example, the inner conductor 72 can form a layer or coating on the entire interior surface of the inner lumen 76 of the dielectric member 70. As such, the inner conductor 72 can be cylindrical or tubular. In yet another example, the inner conductor 72 can form a solid core within the dielectric member 70. Similarly, as shown, the outer conductor 74 can be a relatively straight or linear strip of material that extends between a distal end and a proximal end of the dielectric member 70. Alternatively, the outer conductor 74 can be a strip of material having a nonlinear pattern, such as a zigzag pattern, helical pattern, fractal pattern, back-and-forth pattern, set of radial rings, set of radial bands, or other suitable patterns. Additionally, the inner conductor 72 can be aligned or misaligned axially to result in the desired ablation pattern. At least some of the aforementioned examples are illustrated in FIGS. 4 through 14.

The shape, dimensions, and length of the inner conductor 72 and the outer conductor 74 can work together to tune the antenna element 40 to one or more frequencies. Additionally, to tune the antenna element 40 to the desired frequency or frequency range(s), at least some of the following properties of the antenna element 40 can be adjusted: the dielectric constant of the dielectric member 70, the thickness of the dielectric member 70, the diameter of the dielectric member 70, and the length of the antenna element 40. Each of these properties will be described below.

Referring still to FIG. 3, the properties of the dielectric member 70 can be selected to properly tune the antenna element 40 to the desired frequency. In some embodiments, the dielectric member 70 is a ceramic material. For example, the dielectric member 70 can comprise alumina, silicon nitride, titania, other metal oxides, quartz, and/or other ceramic materials. The dielectric constant of the dielectric member 70 can be between about 4 to about 30 or greater than 30. In some configurations, the dielectric constant of the dielectric member 70 can be between about 9 to 10.5. In some configurations, the dielectric member 70, such as a dielectric member 70 made of alumina, has a dielectric constant of about 9.8. In some configurations, the thickness of the dielectric member 70 is between about 0.001 to 0.05 inches. In some configurations, the thickness of the dielectric member 70 is between about 0.005 to 0.04 inches. In a specific embodiment the thickness is between 0.0001 to 0.03 inches. In some configurations, the diameter of the dielectric member 70 is between approximately 0.01 to 0.15 inches.

The properties, shapes, and dimensions of the inner and outer conductors 72, 74 can be selected to properly tune the antenna element 40 and customize the shape the radiation pattern. For example, in some embodiments the inner and/or the outer conductor 72, 74 form conductive strips. These strips can have a width between about 0.001 inches and 0.1 inches. These strips can have a thickness between about 0.001 inches and 0.05 inches. The width can be less than 0.001 inches and the thickness can be less than 0.001 inches when certain thin film fabrication methods are utilized. Additionally the inner and outer conductors 72, 74 can be made of various conductive materials including conductive metals, inks, composites, and the like. Example materials include copper, tin, aluminum, gold, silver, inconel, brass, degenerate transparent semi-conductors, and the like. Conductive particles can also be applied to the inner and outer conductors 72, 74 or connected to these conductors. In some embodiments, the cross section of these conductors can include multiple extruded conductive metal-to-metal materials to combine the desired physical and/or mechanical attributes. These combined materials may be contained in one outer diameter wire, cable or ribbon to produce optimal radio frequency fields and conductivity.

The antenna element 40 can be manufactured using one or more of a variety of manufacturing processes. For example, the dielectric member 70 can be formed as a dielectric tube that can be inserted over an inner conductor 72 and upon which can be applied an outer conductor 74. For example, the inner and/or outer conductors 72, 74 can be screen-printed using conductive ink or paint onto the dielectric material.

In a specific example, the inner conductor 72 or the outer conductor 74 can comprise a metal ink, such as silver or copper ink. Metal ink can be painted or otherwise applied to the outer surface of the dielectric member 70 using various processes. In some instances, a removable mask, such as tape, is placed in a desired helical pattern of the outer surface of the dielectric member 70. A metal ink is then applied on the exposed surface of the dielectric member 70 either via painting, vapor deposition, or some other application process. The metal ink can be dried, such as in a dryer for about 10 to 30 minutes. In some instances, the removable mask is then removed and the metal ink can be baked, such as in an oven. In other instances, the removable mask is removed after baking. In some embodiments, the metal ink is baked at about 800 to 1100 degrees Celsius for between about 1 to 10 minutes. In some embodiments, a metal powder can be applied to the metal ink before it is dried and/or cured. This metal powder can provide at least some properties of pseudo-fractal antennas, as will be described below.

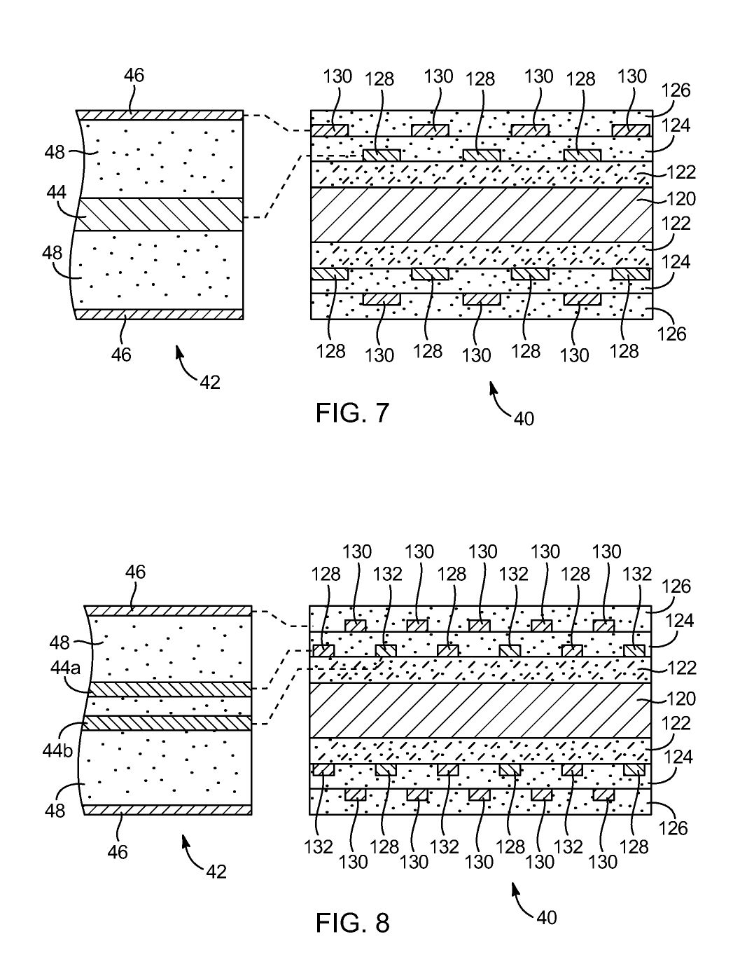

In another example, the inner conductor 72, the dielectric member 70, and/or the outer conductor 74 can be manufactured using a deposition, sputtering, or other growing or coating processes. For example one or more of these structures can be formed using one or more growth processes and/or one or more thin or thick film deposition processes, such as sputtering CVD, or evaporative coating processes. These processes will be discussed in greater detail with reference to FIGS. 7 and 8.

Referring still to FIG. 3, in some embodiments, the antenna element 40 forms a microstrip-type antenna element. Generally, a microstrip-type antenna includes an antenna element pattern in a metal trace bonded to a dielectric substrate, such as a printed circuit board, with a metal layer bonded to the opposite side of the substrate which forms a ground plane. The antenna element 40 shown in FIG. 3 can operate using at least some of the same principles as the aforementioned planar microstrip antenna elements. For example, the inner conductor 72 can function as a ground plane, the dielectric member 70 can function as the dielectric substrate, and the outer conductor 74 can function as the metal trace. In another example, the outer conductor 74 can function as the ground plane and the inner conductor 72 can function as the metal trace. In some configurations, embodiments of a microstrip-type antenna element 40 can be smaller and produce a more spherical radiation pattern than other antenna types

Microstrip-type antenna element 40 can provide a number of advantages to radio frequency ablation procedures. In some embodiments, microstrip antennas can utilize ceramic dielectrics that can be made smaller and are more heat resistant than some other types of dielectrics. Because microstrip-type antenna element 40 can be more heat resistant, they can be driven at higher power levels to produce larger and/or hotter ablation regions with smaller devices. Thus, in some embodiments, microstrip-type antenna elements 40 can create a more controlled temperature pattern than other types of ablation antennas. In some instances, a microstrip-type antenna element's 40 ability to increased and/or vary the power levels, enables a clinician to increase or decrease the power to the microstrip-type antenna element 40 in order to match the size of the ablation zone to size of the target structure 34 (shown in FIG. 1).

FIG. 4 illustrates another example of an antenna element 40 in accordance with some embodiments of the invention. In some embodiments, this antenna element 40 can replace the antenna element 40 shown in FIG. 1 or 2.

As shown, the antenna element is connected to a coaxial cable 42 similar to that shown in FIG. 2. The antenna element 40 can be mechanically and electronically coupled to the distal end of the coaxial cable 42. In some instances, one or more conductors of the coaxial cable 42 continue into the antenna element 40, providing both a mechanical and an electrical connection between these two structures. The connection area 96 between the antenna element 40 and the coaxial cable 42 can also soldered together or joined using an adhesive or other fastener. Moreover, a gap can be provided between the coaxial cable 42 and the antenna element 40 provide electrical separation between these two structures. This gap can be filled using an insulating material or it can be left open. Other means for connecting the antenna element 40 to a coaxial cable are contemplated.

The antenna element 40 can include a dielectric member 70 that has a cylindrical tube shape. An inner conductor 80 can be disposed within the dielectric member 70 and form a solid core therein. The inner conductor 80 can be connected to the outer conductor 82 of the coaxial cable 42, which can be connected to ground. The outer conductor 82 can be wrapped around the outside of the dielectric member in a spherical or helical pattern, as shown. The outer conductor 82 can be connected to the inner conductor 80 of the coaxial cable 42, which can be connected to the radio frequency power source 26 (shown in FIGS. 1 and 3). By wrapping the outer conductor 82 around the outer surface of the dielectric member 70 the overall length 90 of the antenna element 40 can be much shorter than the overall length of the outer conductor 82. As such, the overall length 90 of the antenna element 40 can be relatively small and contribute to the production of a more spherical radiation pattern. This can be because a shorter antenna element 40 can respond more similarly to a theoretical point source antenna having a substantially spherical radiation pattern. Moreover, the length of the outer conductor 82 can be an integer multiple of a quarter wavelength (e.g., a quarter wavelength, a half wavelength, a full wavelength, or the like) of the desired transmission frequency of the radio frequency power source 28.

In a non-limiting example, the outer conductor 82 can have a length of about 2-inches long and be wrapped around an aluminum oxide dielectric. This length can be impedance matched to wet tissues, such as at 915 MHZ. In other instances, the length is impedance matched to other frequencies in the microwave band or in another band.

Generally, the antenna element 40 of FIG. 4 can function as a helical microstrip-type antenna element in which the inner conductor 80 functions as a ground plane and the outer conductor 82 functions as the antenna trace element.

As mentioned, the various dimensions, configurations, and materials of the antenna element 40 can be selected to tune the antenna to the desired frequency and power levels. As mentioned, the antenna element 40 can be configured to transmit one or more microwave frequencies. To tune the antenna element 40 of FIG. 4 to the desired frequency(ies) and/or to the impedance of the desired tissue(s), at least the following properties of the antenna element 40 can be adjusted: the dielectric constant of the dielectric member 70, the thickness 92 of the dielectric member 70, the diameter 94 of the dielectric member 70, the number of winds of the outer conductor 82, the thickness 84 of the outer conductor 82, the width 86 and length of the outer conductor 82, the spacing 88 between winds of the outer conductor 82, the dimensions of the inner conductor 80, and the length 90 of the antenna element 40. These properties will be described below.

The properties of the dielectric member 70 can be selected to properly tune the antenna element 40 to the desired frequency. In some embodiments, the dielectric member 70 is a ceramic material. For example, the dielectric member 70 can comprise alumina, quartz, or other ceramic materials. This dielectric member 70 can be tube-shaped and be inserted over the inner conductor 80 that forms the ground plane. In some configurations, the dielectric constant of the dielectric member 70 can be between about 4 to about 30 or greater than 30. In some configurations, the dielectric constant of the dielectric member 70, such as alumina, can be between about 9 to 10.5. In some configurations, the dielectric member 70 has a dielectric constant of about 9.8. In some configurations, the thickness 92 of the dielectric material is between about 0.002 to 0.04 inches. The thickness can be less than 0.002 inches when certain thin film fabrication methods are utilized. In some configurations, the thickness is about 0.1 inches. In some configurations, the diameter 94 of the dielectric member 70 is between approximately 0.001 to 0.25 inches.

The properties of the outer conductor 82 can also be selected to properly tune the antenna element 40 and customize the shape the radiation pattern. As shown, the outer conductor 82 can be disposed around the dielectric member 70 in a helical or spiral pattern. The properties of the outer conductor 82 and the winding properties can affect radiation pattern. Thus, in some embodiments, the outer conductor 82 is wound tightly (having a narrow spacing 88 between adjacent windings) and close so that the length 90 of the antenna element 40 is small and the radiation pattern is substantially spherical. In some configurations, the outer conductor 82 comprises a strip of conductive material having a width 86 between about 0.001 inches and 0.25 inches. In some configurations the thickness 84 of the outer conductor 82 is less than or equal to 0.004 inches. The number of winds can range between 0.5 to 50 winds. In some embodiments, there are between about 0.5 to 20 winds. In some embodiments, there are between about 1 to 15 winds. The spacing 88 between winds of the outer conductor 82 can be between about 0.001 to 0.1 inches. In some instances, the spacing 88 is between about 0.001 to 0.07 inches. Each of the properties of the outer conductor 82 can affect the length, he of the antenna element 40. In some instances, the length 90 is between about 0.1 inches to 1.0 inch. In some instances, the length is about 0.5 inches. Other configurations can include a length between 1 and 3 inches for larger ablation area.

In a particular embodiment, the antenna element 40 is configured to transmit at a frequency of about 915 MHz at about between 90 W to 180 W of power. The antenna element 40 can have the following specific dimensions: The dielectric member 70 can be a 0.05 inches alumina tube with a dielectric constant of about 9.8. The outer diameter 94 of the dielectric member 70 is between about 0.09 to 0.125 inches. The inner diameter of the dielectric member 70 is between about 0.011 to 0.02 inches. The thickness 92 of the dielectric member 70 is about 0.039 inches. The outer conductor 82 has about twelve winds that span between about 0.05 to 0.09 inches. The spacing 88 between the winds is between about 0.01 to 0.037 inches. The width 86 of the outer conductor 82 is about 0.035 inches.

As further shown in FIG. 4, the antenna element 40 can optionally include an end cap 98 at its distal end. The end cap 98 can be made of a conductive material (e.g., a metal) or an insulating material. The end 98 can affect the shape and direction of the radiation pattern by decreasing its length (dimension along the longitudinal axis of the antenna element 40). Thus, in some instances, the end cap 98 can make the radiation pattern more spherical, and at least partially preventing it from being directed out the distal end. In some configurations, the end cap 98 is not electrically coupled to either the inner conductor 80 or the helical conductor 54 but insulated from both these structures. In some instances, the end cap 98 is coupled only to the dielectric member 70. In some other instances, the end cap 98 can be coupled to a grounded conductor, such as the inner conductor 80 shown in FIG. 4. Thus, the end cap 98 may not be coupled to the outer conductor 82 or another conductor that is connected to the radio frequency power source.

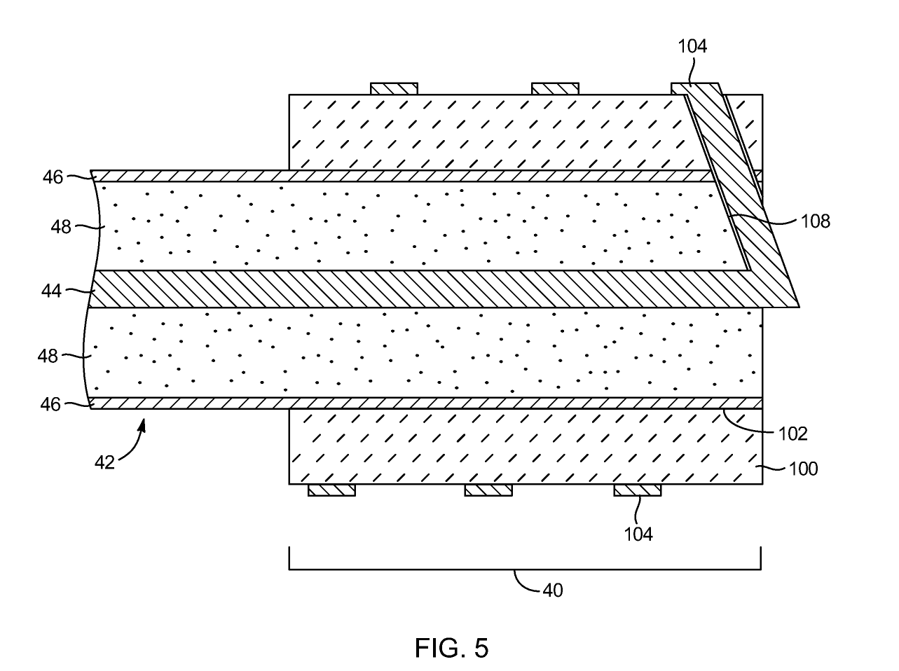

FIGS. 5-9 depict other examples of antenna elements 40. It will be understood that while these examples illustrate antenna elements 40 having different configurations, many of the properties structures, and features can be the same or similar to those described with reference to FIGS. 3 and 4. For example, the number of winds, the spacing between the winds, the dielectric material with its circumference and thickness, and/or the width and height of the outer conductor 82, etc. can be varied and previously mentioned.

Referring now to FIG. 5, an antenna element 40 is shown having a dielectric member 100 that circumscribes a distal portion of the exterior of the coaxial cable 42. In some embodiments, this antenna element 40 can replace the antenna element 40 shown in FIG. 1 or 2.

As shown, the outer conductor 46 of the coaxial cable 42 forms the inner conductor 102 of the antenna element 40 over the length of the antenna element 40. The inner conductor 102 can be bonded to or otherwise coupled to the dielectric member 100. As in the example antenna element 40 of FIG. 4, an outer conductor 104 can be disposed on the outer surface of the dielectric member 100 in a helical, spherical or other pattern. The inner conductor 102, as part of the outer conductor 46 of the coaxial cable 42, can be connected to ground. The outer conductor 104 can be connected to the inner conductor 44 of the coaxial cable 42, which can be connected to the radio frequency power source. As shown, a cutout groove 108 can be formed in the distal end of coaxial cable 42 to accommodate an electrical connection between the inner conductor 44 of the coaxial cable 42 and the outer conductor 102 of the antenna element 40.

In some embodiments, the configuration of FIG. 5 can provide a shorter antenna element 40 than that of FIG. 4 because the outer diameter of the dielectric member 100 is larger and thus has a larger circumference. Thus, the outer conductor 104 can have the same length for antenna tuning purposes but have fewer winds. Thus, the antenna element 40 can have a shorter length. In some configurations, the shorter length can act more like a point source and can provide a more spherical radiation pattern.

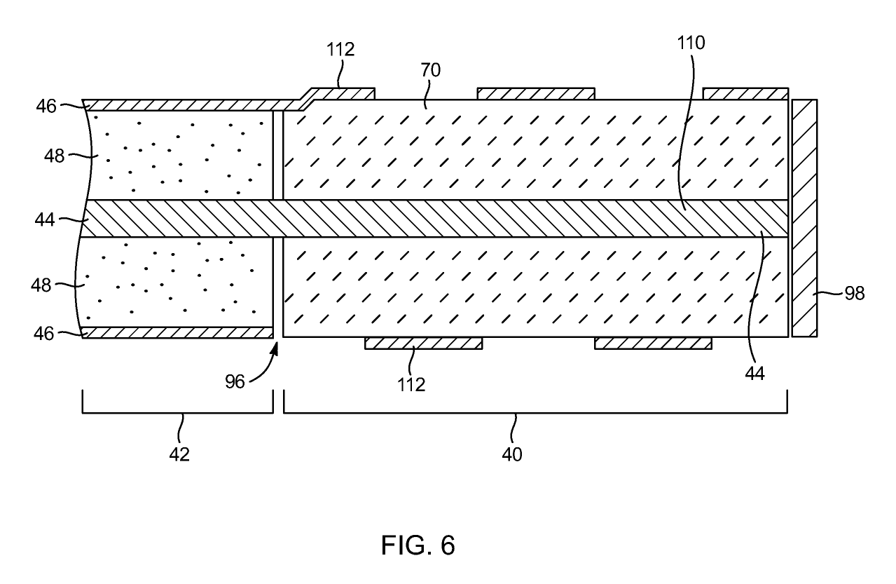

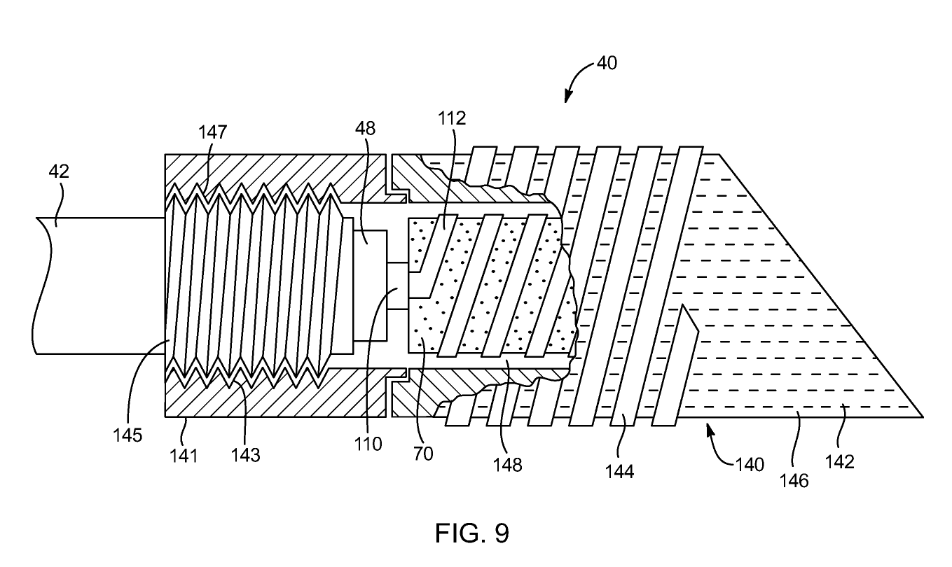

Reference will now be made to FIG. 6, which depicts another example of an antenna element 40. In some embodiments, this antenna element 40 can replace the antenna element 40 shown in FIG. 1 or 2. FIG. 6 depicts a similar antenna element to that of FIG. 4, and the properties of the individual components, dimensions, shapes, and sizes of the individual components can be similar to those described with reference to FIG. 4. In other embodiments, as shown in FIG. 9, a separate sleeve can also placed over the antenna element 40, as described with reference to that Figure.

As shown in FIG. 6, the antenna element 42 is similar to the antenna element 40 of FIG. 4, with the exception that the inner conductor 110 of the antenna element 40 can be an extension of or is connected to the inner conductor 44 of the coaxial cable 42. Moreover, the outer conductor 112 of the antenna element 40 can be connected to the outer conductor 46 of the coaxial cable 42. Thus, when this antenna element 40 is functioning as a microstrip type antenna element, the outer conductor 104 functions as the ground plane, and the inner conductor 102 functions as the microstrip trace. While the outer conductor 112 functions as a ground plane, it may still be disposed in a helical or spherical pattern about the exterior of the dielectric member 70, which can permit radiation to propagate through the spaces between the windings. Other patterns of the outer conductor 112 are also contemplated. In these configurations, the antenna element 40 may function as a slot antenna using inside and outside helical wrap. The transmitted energy can pass between the gaps in the outer conductor 112.

As configured in FIG. 6, the feed line signal is carried into the center of the antenna element 40 rather than around the exterior of the antenna element. In some configurations, the feed line signal is carried into the center of the antenna element and can be wrapped around a smaller dielectric member 70. The smaller dielectric member 70 can have for example about a 0.050 inch diameter.

Reference will now be made to FIGS. 7 and 8, which depicts another example of an antenna element 40 in accordance with some embodiments of the invention. In some embodiments, each of these antenna elements 40 can separately replace the antenna element 40 shown in FIG. 1 or 2. These examples illustrate an antenna element 40 that can be manufactured using a deposition, sputtering, or other growing or coating processes. For example one or more of these structures can be formed using one or more growth processes and/or one or more thin or thick film deposition processes, such as sputtering, CVD, or evaporative coating processes. Additionally, these antenna elements 40 can be connected to a coaxial cable 42 as previously described and shown with reference to FIGS. 4 through 6. Moreover, other forms of connecting the antenna element 42 to a coaxial cable 42 are contemplated.

As shown, the antenna element 40 can include a dielectric member 124, an inner conductor 128, and an outer conductor 130. As further shown, the antenna element 40 can optionally include a support rod 120, a support layer (e.g., an oxide layer or the like) 122 formed on the support rod 120, and/or an outer dielectric layer 126 formed on the exterior of the dielectric member 124 and the outer conductor 130.

As mentioned, the antenna element 40 of FIGS. 7 and 8 can be formed using one or more growth processes and/or one or more thin or thick film deposition processes. While this type of manufacturing is described with reference to the embodiments of FIGS. 7 and 8, these same processes can be used to form each of the other antenna elements embodiments shown in FIGS. 3 through 17. A representative example of these processes will now be described.

As shown, a support rod 120 can be provided upon which can be grown or deposited the components and structures of the antenna element 40. The support rod 120 can have various lengths, for instance, lengths between about 0.040 to 2.0 inches, preferably 0.04 to 0.5 inches. The support rod 120 can be anodized so that its outer surface is oxidized to form a supporting layer 122. The inner conductor 128 can be deposited on the support rod 120 or the support layer 122. The material of the inner conductor 128 can be deposited using a sputtering or other such process. The inner conductor 128 can be formed into a certain trace pattern, such as a helical pattern, using lithography and etching processes or other such processes. In other embodiments, the support rod 120 can be conductive and be used as the inner conductor 128. As such, an inner conductor 128 may not need to be deposited on the support rod 120.

After the inner conductor is provided, as mentioned above, the dielectric material 124 (e.g., silicon nitride) can be grown or deposited over the exposed portions of the support layer 122 and the inner conductor 128 to form the dielectric member 124. The outer conductor 130 can then be formed on the outer surface of the dielectric member 124 using similar processes used to form the inner conductor 128. The conductive layer of the inner conductor 128 and the outer conductor 130 can be between about 10 to 300 nanometers. Optionally, another dielectric layer 126 can be grown, deposited, or otherwise formed on the exposed portions of the dielectric member 124 and the outer conductor 130. The dielectric member 124 can have a thickness between about 10 to 300 nanometers, including between about 20 to 50 nanometers. The overall diameter of the antenna element can be between about 0.01 inches and 0.125 inch.

As further shown in FIG. 7, the inner conductor 128 can be connected to the inner conductor 44 of the coaxial cable 42, and the outer conductor 130 can be connected to the outer conductor 46 of the coaxial cable 42. These connections can also be reversed such that the inner conductor 128 is connected to the outer conductor 46 of the coaxial cable 42, and the outer conductor 130 is connected to the inner conductor 44 of the coaxial cable 42. As previously discussed, the dimensions of the inner conductor 128 and the outer conductor 130 as well as the number of windings and spacing between the windings can be configured and otherwise selected to tune the antenna to the desired frequency(ies) and/or to the impedance of the desired tissue(s).

Reference will now be made to FIG. 8, which illustrates an antenna element 40 that is similar to the antenna element 40 of FIG. 7 except that it has a second inner conductor 132 (which is a third conductor). Both the first inner conductor 128 and the second inner conductor 132 can be helically wrapped around the supporting rods 120 and disposed on an inner surface of the dielectric member 124. It will be understood that in other instances, the antenna 40 can also include a third or fourth inner conductor (not shown) that employ the same principles of the second inner conductor 132. Similarly, it will be understood that in other instances the antenna element 40 can have a second outer conductor, third outer conductor, or fourth outer conductor (not shown), which employ the same principles of the second inner conductor 132.

As shown, the first inner conductor 128 can be connected to a first inner conductor 44a of the coaxial cable 42, and the second inner conductor 132 can be connected to a second inner conductor 44b of the coaxial cable 42. Referring to both FIGS. 2 and FIG. 8, the controller 28 of the radio frequency power source 26 can be configured to control the phase of energy delivered to the first inner conductors 128 and second inner conductor 132. Thus, the controller 28 can create a phase differential between the two separate signals transmitted on the first inner conductor 128 and the second inner conductor 132. Similarly, in instances where a third and/or a fourth inner conductors are added to the antenna element 40 of FIG. 8 the controller 28 can be configured to transmit energy having a different phase to each of these conductors.

The use of a multiple phase antenna element, such as the two-phase antenna element 40 of FIG. 8, or a three-phase antenna element (not shown) can be used to manipulate the size and shape of emitted radiation patterns and consequently the ablation regions. Thus, relative phases can be manipulated so that the ablation regions can be shaped to be the same size as a target structure 34 or so that they can be directed toward a target structure near a probe member 20 (shown in FIG. 1). Using this functionality, the ablation regions may be moved distally, proximally, or axially about the probe member 20. Such manipulability and directionality can be produced, in some instances, by varying the phase between transmitted radio frequency energy transmitted through multiple conductors of the antenna element 40, as shown in FIG. 8.

While the use of two or more conductors that can be provided with signals having different phases is described and illustrated only with reference to FIG. 8, these structures and features can be used with any other antenna element embodiments of FIGS. 2 through 16. As such, the single inner conductor or outer conductor of these Figures can be replaced with two, three, or more separate conductors, each configured to transmit a separate signal.

Reference will now be made to FIG. 9, which depicts another example of an antenna element 40. In some embodiments, this antenna element 40 can replace the antenna element 40 shown in FIG. 1 or 2. Similar to previously described antenna devices, this device may stem from a coaxial cable 42. The antenna element 40 can comprise an inner portion that can have the same configurations as those illustrated in FIGS. 4 to 6 and described herein. As shown, the inner portion is similar to that shown in FIG. 6 and described with reference to that Figure.

As shown, the antenna element 40 includes a sleeve 140 that is selectively disposed over the antenna element 40 and coupled to the coaxial cable 42 via, for example, a set of threads 143, 147 or other like adjustable connectors, such as brass sleeves that can be press fitted on and rotated and soldered in place without threads. The sleeve 140 may be rotationally adjustable about the longitudinal axis (extending along its length) of the coaxial cable 42 and/or axially adjustable along the longitudinal axis of the coaxial cable 42. The sleeve 140 can include a connector portion 141 and an antenna portion 146. These two portions can be coupled together, such as with a solder or a weld, which can include a thermal adhesion bond. This coupling can be assisted by adding copper or silver ink to the entire proximal end of the antenna portion. The connector portion 141 selectively connects the sleeve 140 to the coaxial cable 42. The antenna portion 146 can include antenna components used to interact with the radiation emitted from the antenna element 40 to modify the emitted radiation pattern in a manner that produces a desired radiation pattern. In some configurations, the antenna portion 146 includes a dielectric tube 142 or sleeve that covers and at least substantially encloses the antenna element 40 therein. To encourage electronic isolation, a gap 148 can be configured between the dielectric tube 142 and the antenna element 40. This gap 148 can be maintained during both storage and use. The dielectric tube 142 can include one or more conductors 144 disposed thereon. The one or more conductors 144 can be conductive traces and can have various configurations, such as those described herein, including a helical configuration. The one or more conductors 144 can be connected to a radio frequency power source, ground, or are free standing.

To provide adjustability to the adjustable sleeve 140, the outer portion of the coaxial cable 42 can include threads 145. These threads 145 can be manufactured as part of the coaxial cable 42 or be installed thereon after the manufacture of the coaxial cable 42. These threads 145 can be brass or copper threads or made of another type of rigid or semi-rigid material. The threads 145 can be male threads, as shown, or other thread types. In some configurations, the sleeve 140 is selectively coupled to the coaxial conductor 42 via the threads 145. The sleeve 140 can also includes a threaded connector portion 141 that includes threads 143, such the illustrated female threads. In other embodiments, other adjustable components are disposed between the coaxial cable 42 and the sleeve 140 that enable the sleeve 140 to be coupled over the coaxial cable 42 at various locations on the adjustable sleeve 140.

By adjusting the distance to which the sleeve 140 is threaded onto the coaxial cable 42 a manufacturer or user can tune the antenna element 40 to certain frequencies. In some instances, a manufacturer may properly tune the adjustable sleeve and then fixedly couple (e.g. via soldering mechanical, thermo bonding, and/or other like processes) the adjustable sleeve 140 in place. As the sleeve 140 is advanced over the threads 145, it is also rotated. These movements can change the frequency response of the antenna element 40. In some embodiments, the antenna device is configured to have very low or approximately no reflected power during the ablation process. With the sleeve 140 disposed over the antenna element 40, the resulting radiation pattern can be affected which can adjust the shape and/or size of the resulting radiation pattern. Thus, the dielectric tube 142 and the outer conductor 144 of the sleeve 140 can function with the inner antenna element 40 to serve as a combined antenna element. This configuration can provide a short antenna element that can produce spherical or nearly spherical ablation pattern when properly tuned. It will be understood, that the interface between threads of the sleeve 140 and threads 145 on the coaxial cable can be tight enough to allow the sleeve 140 to remain in a fixed position after it is threaded a certain distance while also be loose enough to allow the sleeve 140 to be adjusted as needed.

In some embodiments, a fixed sleeve (not shown) is used in place of the adjustable sleeve. The fixed sleeve can be mechanically and/or electrically coupled to the coaxial cable 42. The fixed sleeve can have an antenna portion 146 similar to that of the adjustable sleeve 140. The fixed sleeve can be fixed in a position and orientation in which the antenna device is tuned to a desired frequency or frequency range.

The various dimensions and proportions of the antenna element 40, the dielectric tube 142, the outer conductor 144, the gap 148, and other components can be shaped and sized to produce the desired radiation pattern, as will be understood and as described herein.

Additionally, the distal end of the dielectric tube 142 can be shaped and sized to produce an angled edge or point, as shown. This distal end can be used as a needle head for piercing through flesh or other bodily features. In some instances, this distal end can be reinforced, isolated, and/or insulated via a coating, a protective cover, or other member.

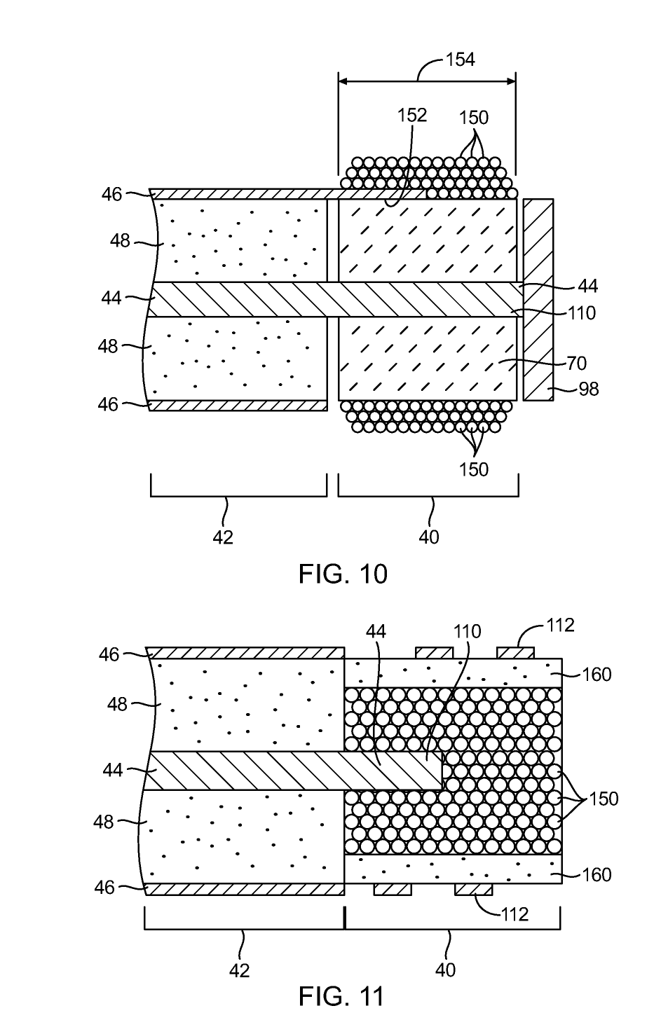

FIG. 10 illustrates another example of an antenna element 40, which has a plurality of conductive particles 150 disposed on the outer surface of the dielectric member 70. In some embodiments, this antenna element 40 can replace the antenna element 40 shown in FIG. 1 or 2. Similar to previously described antenna elements, this antenna element 40 can be connected to a coaxial cable 42 through which it is connected to a radio frequency power source and/or ground.

As shown, the inner conductor 44 of the coaxial cable 42 can extend into the antenna element 40 to form the inner conductor 110 of the antenna element 40. A dielectric tube and 70 can be disposed about the inner conductor 110, and an end cap 98 can optionally be disposed and/or coupled onto the distal end of the antenna element 40. The dimensions and properties of the aforementioned complements can be similar to those described with reference to the embodiments of FIG. 4. The outer conductor 46 of the coaxial cable 42 can have at least a portion thereof that extends onto the outer surface of the dielectric tube 70 of the antenna element 40 to form an outer conductor 152. The outer conductor 152 can form an electrical contact with a plurality of conductive particles 150 that are disposed on the outer surface of the dielectric tube 70. The plurality of conductive particles 150 can be used to affect the radiation pattern of the antenna element 40. In other embodiments, the inner conductor 44 of the coaxial cable 42 can be connected to the outer conductor 152 of the antenna element 40, and the outer conductor 46 of the coaxial cable 42 can be connected to the inner conductor 110 of the antenna element 40.

In some embodiments, the plurality of conductive particles 150 function similar to a fractal antenna, thus being referred to herein as a pseudo-fractal antenna. A fractal antenna is an antenna that uses a fractal design, or a self-similar design, to maximize the length or perimeter of material that can receive or transmit electromagnetic radiation within a given total surface area or volume. In some instances, the plurality of conductive particles 150 has at least some self-similar designs, shapes, and sizes, that increase the perimeter of the antenna element 40, permitting the antenna element 40 to have a shorter length 154 and to provide a more spherical radiation pattern. Because a fractal antenna's response is capable of operating with good-to-excellent performance at many different frequencies simultaneously, the fractal-nature of the plurality of the pseudo-fractal conductive particles 150 can also improve the antenna element's performance and tune-ability.

The plurality of conductive particles 150 can be small particles of various types of conductive metals. In some embodiments, the plurality of conductive particles 150 can comprise at least one of aluminum, copper, silver, other conductive particles, or combinations thereof. The size of the conductive particles 150 can be between about 100 to 320 Mesh (about 150 to 40 microns). In other embodiments, the size of the conductive particles 150 is between about 50 to 625 Mesh (about 300 to 20 microns). In other embodiments, the size of the conductive particles 150 is between about 250 to 300 Mesh (about 105 to 74 microns).

In some instances, the plurality of conductive particles 150 can be bound together using a binding member. The binding member can be an adhesive, a metal ink, or another conductive binding member. For example, a metal ink can be applied to the outer surface of the dielectric member 70. Next, the portion of the dielectric member 70 having the wet metal ink can be dipped into a container having a plurality of conductive particles 150, which adhere to the metal ink. The dielectric member 70, the metal ink, and the plurality of conductive particles 150 can be cured. In some configurations, curing takes place in an oven at about 500 degrees Celsius for about 15 minutes. Other curing procedures can also be used. In other instances, the plurality of conductive particles 150 are partially melted, such adjacent particles bind together without a binding member.

FIG. 11 illustrates an example of an antenna element 40, which has a plurality of conductive particles 150 disposed within a dielectric member 160. In some embodiments, this antenna element 40 can replace the antenna element 40 shown in FIG. 1 or 2. Similar to previously described antenna elements, this antenna element 40 can be connected to a coaxial cable 42 through which it is connected to a radio frequency power source and/or ground. This antenna element 40 can be used to direct a radiation pattern outwardly from the distal tip of the antenna element 40 along the longitudinal axis of the probe member 20 (shown in FIGS. 1 and 2).

As shown, the antenna element 40 includes a dielectric member 160 in the shape of a cylindrical tube. An outer conductor 112 is disposed on the outer surface of the dielectric member 160. The outer conductor 112 is connected to the outer conductor 46 of the coaxial cable 42. The inner conductor 110 of the antenna element 40 is an extension of or is connected to the inner conductor 44 of the coaxial cable 42. The inner conductor 110 is electronically coupled to a plurality of conductive particles 150, which are disposed within the dielectric member 160. The plurality of conductive particles 150 can be used to affect the radiation pattern of the antenna element 40, as described with reference to the antenna element 40 of FIG. 10. Moreover, in some embodiments, the antenna element 40 includes an end cap that assists to retain the conductive particles 150 within the dielectric member 160. In other embodiments, the antenna element 40 can be hermetically sealed in order to retain the conductive particles 150 within the dielectric member 160.

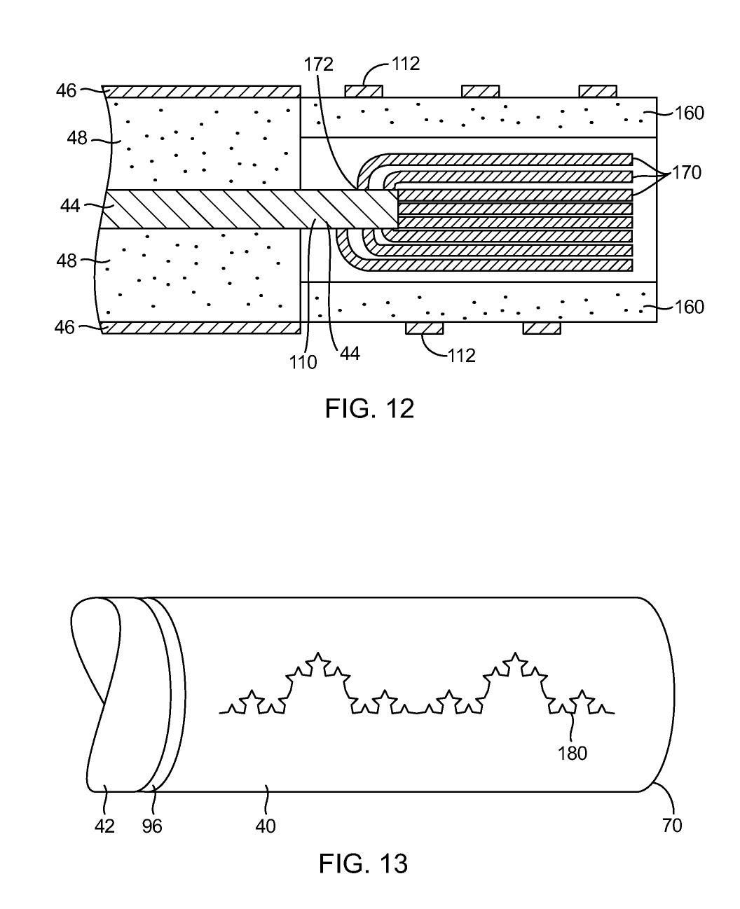

FIG. 12 illustrates an example of an antenna element 40, which has a plurality of conductive wires 170 disposed within a dielectric member 160. In some embodiments, this antenna element 40 can replace the antenna element 40 shown in FIG. 1 or 2. Similar to previously described antenna elements, this antenna element 40 can be connected to a coaxial cable 42 through which it is connected to a radio frequency power source and/or ground.

The antenna element 40 of FIG. 12 can be similar to antenna element of FIG. 11, except that the plurality of conductive particles can be replaced by a plurality of conductive wires 170. The conductive wires 170 can include fine/small wire strands, fibers, or other miniaturized elongated conductive structures. Such wires can have a relatively small thickness, such as between about 1-10 millimeters. Some of the wires could be part of the inner conductor 44 of a coaxial cable 42, which extend to the antenna element 40. The use and function of the conductive wires 170 can be similar to that of the conductive particles in that they similarly affect the radiation pattern of the antenna element 40. As shown, the conductive wires 170 can be aligned along the longitudinal axis of the antenna element 40. Additionally and/or alternatively, the conductive wires 170 can be folded over each other, wrapped together, tied together, or otherwise inserted in an orderly or disorderly fashion within the dielectric member 160. The conductive wires 170 can be coupled to the inner conductor 110 using a coupling 172 which can be a mechanical chemical or other such coupling device.

As further shown, some of the plurality of conductive wires 170 can have different lengths. The different lengths the wires and help stabilize the frequency range and the overall impedance the antenna element 40. For example, the standing wave reflected power throughout the ablation process may need to be kept at about 50 ohms, which may be achieved using the different lengths of wire. These lengths can be between about 0.1 to 4 inches, about 1.3 to 3 inches, or about 0.5 to 2.5 inches. Additionally, the diameter or each wire can vary as well.

In some embodiments, the antenna element 40 of FIG. 12 can include an end cap that assist to retain the conductive wires 170 within the dielectric member 160. In other embodiments, the antenna element 40 can be hermetically sealed in order to retain the conductive wires 170 within the dielectric member 160.

FIG. 13 illustrates an example of an antenna element 40 that has an outer conductor 180 disposed in a fractal pattern on the outer surface of the dielectric member 70. In some embodiments, this antenna element 40 can replace the antenna element 40 shown in FIG. 1 or 2. Moreover, the fractal pattern can replace the helical pattern shown in prior Figures. In some embodiments, this and other configurations of fractal patterns can replace the helical patterns of the antenna elements illustrated in FIGS. 4 to 6. In some instances, the fractal pattern can be wrapped around the outer surface of the dielectric material in various fashions, such as in a semi-helical fashion.

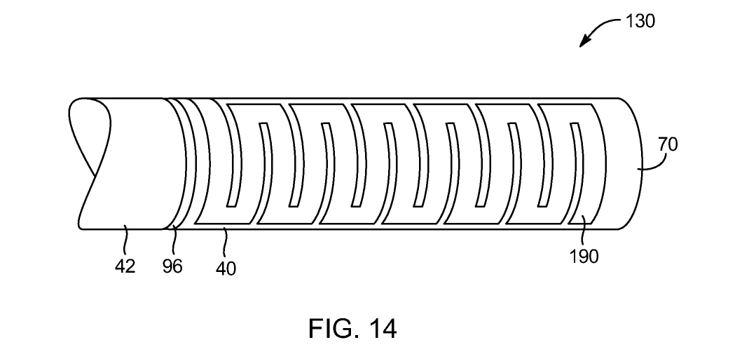

FIG. 14 illustrates an example of an antenna device 40 that has an outer conductor 190 disposed only one a portion of the outer surface of the antenna element 40. In some embodiments, this antenna element 40 can replace the antenna element 40 shown in FIG. 1 or 2. Moreover, in some embodiments, this and other antenna patterns or other like antenna patterns can replace the helical patterns of the antenna elements illustrated in FIGS. 4 to 6. In other embodiments, outer conductor 190 is disposed around only one quadrant, two quadrants, three quadrants, and/or portions of a quadrant of the dielectric member 70. These configurations can enable the antenna element 40 to be configured to provide a uniform radiation pattern about the entire antenna element 40 or to provide a customized or directional radiation pattern.

Reference will now be made to FIGS. 15 and 16, which illustrate examples of an antenna element 40 formed using a dielectric member 200 having a relatively flat configuration, as opposed to a tubular configuration. In some embodiments, these antenna elements 40 can each separately replace the antenna element 40 shown in FIG. 1 or 2. Moreover, aside from having relatively flat or planar members, these antenna elements can include the same features, materials, thicknesses, etc. as those antenna element embodiments previously described.

As shown, the antenna element 40 can be planar rather than cylindrical or tubular. In other embodiments, the antenna element 40 can have other non-circular cross sections, such as square, triangular, or other polygon cross-sections. Additionally, the antenna element 40 can have other shaped cross-sections and non-uniform cross sections over the length of the antenna device. As shown, the antenna element 40 can include a first conductor 204, a dielectric 202, and a second conductor 202. In some embodiments, the first conductor 204 is a ground plane connected to ground and the second conductor 202 is a microstrip trace connected to a radio frequency power source (e.g., radio frequency power source 26 of FIG. 1). In other embodiments, the second conductor 202 is a ground plane and the first conductor 204 is a connected to a radio frequency power source. In some embodiments, the dielectric 200 has a dielectric constant of between about 4 and about 30.

Reference will now be made to FIG. 16, which depicts other embodiments of an antenna element 40. As shown, in some embodiments, the antenna element 40 can include a stacked set of components. For instance, the antenna element 40 can include a set of conductors which are disposed between a set of dielectric substrates, as shown. The depicted antenna element 40 includes a stack of material comprising, in order, a first conductor 202, a first dielectric 200, a second conductor 204, a second dielectric 210, and a third conductor 212. In some configurations, the second conductor 204 can be a ground plane and the first conductor 202 and the third conductor 212 can be a microstrip trace. Alternatively, the first conductor 202 and the third conductor 212 can sever a ground plane and the second conductor 204 can be coupled to the feed signal. In some embodiments, the first and second dielectrics 200, 210 have a dielectric constant of between about 4 and about 30.

Reference will now be made to FIG. 17, which illustrates antenna element 40 configured as a helical dipole antenna. In some embodiments, this antenna element 40 can replace the antenna element 40 shown in FIG. 1 or 2.

In some embodiments, the antenna element 40 of FIG. 17 can be configured to produce a substantially spherical radiation pattern. The antenna element 40 can include two conductors: a first conductor 232 and a second conductor 234. One of these conductors can be coupled to ground while the other is coupled to a feed line. In some embodiments, the first conductor 232 is coupled to ground, while in other embodiments the second conductor 234 is coupled to ground. The antenna element 40 includes a first helical portion 236 and a second helical portion 238. The first and second conductors 232, 234 are disposed substantially parallel to each other and to a longitudinal axis 242 through the center of the first helical portion 236. At a center point 240, the first conductor 232 diverts and forms a coil that winds around the parallel portions of the first and second conductors 232, 234 and the longitudinal axis 242 in the first helical portion. At the center point 240, the second conductor 234 diverts and forms a coil that is winds around the longitudinal axis 242 in the opposite general direction to that of the first conductor 236 in the second helical portion. In this manner, the first and second conductors 232, 234 are maintained with a region of space that is substantially tubular, thus permitting the first and second conductors 232, 234 to be inserted into a probe member 20, such as that shown in FIG. 1.

The antenna element 40 of FIG. 17 can include components, dimensions, and properties that configure the antenna element 40 to transmit microwave energy and to produce ablation-level temperatures that ablate adjacent tissue. In some embodiments, a dielectric material (not shown) is disposed within and about the antenna element 40. In other embodiments, the antenna element 40 includes a cooling system. In some embodiments, the number of winds, the dimensions of each wind, the space between winds, the thickness of each conductor, and/or the dielectric constant of a dielectric material is configured to produce the desired transmission properties. In other embodiments the helical wraps and dielectric insulators can also be applied by thin film deposition methods such as RF magnetron sputtering, evaporative ion coating and chemical vapor deposition or other methods. Materials used for dielectric insulators can include aluminum oxide and/or silicon nitride. Helical wraps can be made of aluminum silver, nickel, and/or copper.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

We claim:

1. A radio frequency ablation (RFA) device comprising:
a dielectric member;
a first conductor disposed within the dielectric member; and
a second conductor disposed on an outer surface of the dielectric member, wherein:
the first conductor is configured to be electrically connected to one of a radio frequency source or ground, and
the second conductor is configured to be electrically connected to the other of the radio frequency source or the ground.

2. The device of claim 1, further comprising a probe member, wherein the dielectric member is disposed within a distal portion of the probe member.

3. The device of claim 2, further comprising one or more sensors connected to the probe member and configured to sense at least one or more of temperature, conductivity, and moisture in proximity to the one or more sensors.

4. The device of claims 2, further comprising a cooling system disposed within the probe member, the cooling system having one or more cooling tubes, the one or more tubes configured to retain a liquid flowing therein.

5. The device of claim 2, further comprising a cooling system disposed within the probe member, the cooling system having one or more a heat pipe, heat transfer conduction pipe, and baffle return system.

6. The device of claim 2, wherein the dielectric member is connected to a distal end of a coaxial cable, the coaxial cable disposed at least partially within the probe member.

7. The device of claim 6, wherein the dielectric member circumscribes at least a portion of a distal end of the coaxial cable.

8. The device of claim 1, wherein the dielectric member has a dielectric constant between about 4 and about 30.

9. The device of claim 1, wherein the first conductor is connected to the radio frequency feed source.

10. The device of claim 1, wherein the first conductor is connected to the ground.

11. The device of claim 1, wherein the second conductor is disposed in a helical pattern on the outer surface of the dielectric member.

12. The device of claim 1, wherein the second conductor is disposed in a fractal or pseudo-fractal pattern on the outer surface of the dielectric member.

13. The device of claim 1, wherein the first conductor is disposed in a helical pattern.

14. The device of claim 1, further comprising:
a third conductor disposed on the outer surface of the dielectric member, wherein the second conductor is electrically coupled to the radio frequency source, and wherein the third conductor is electrically coupled to the radio frequency source; and
a controller for adjusting a phase differential between radio frequency signals transmitted on the second conductor and on the third conductor.

15. The device of claim 1, further comprising:
a third conductor disposed within the dielectric member, wherein the first conductor is electrically coupled to the radio frequency source, and wherein the third conductor is electrically coupled to the radio frequency source; and
a controller for adjusting a phase differential between radio frequency signals transmitted on the first conductor and on the third conductor.

16. The device of claim 1, wherein the second conductor is electrically coupled to a plurality of conductive particles.

17. The device of claim 1, wherein the first conductor is electrically coupled to a plurality of conductive particles disposed within the dielectric member.

17. The device of claim 1, wherein the first conductor is electrically coupled to a plurality of conductive wires of different lengths disposed within the dielectric member.

18. The device of claim 1, further comprising a sleeve adjustably coupled to a coaxial cable, the sleeve being rotationally adjustable about a longitudinal axis of the coaxial cable and axially adjustable along the longitudinal axis of the coaxial cable.

19. The device of claim 18, wherein the sleeve further comprises a dielectric tube having one or more conductors disposed on an outer surface of the dielectric tube.

20. The device of claim 19, further comprising a gap disposed between the sleeve and the outer surface of the second conductor.

21. The device of claim 1, wherein the radio frequency source is configured to provide sufficient power to the first conductor or the second conductor to create sufficient heat to ablate tissues in proximity to the first conductor or the second conductor.

22. The device of claim 1, wherein the radio frequency source is configured to provide radio frequency power having a frequency in the microwave range to the first conductor or the second conductor.

23. A method for manufacturing a radio frequency ablation (RFA) antenna, the method comprising:
providing an inner conductor;
depositing a layer of dielectric material on the exterior of the center conductor, the layer of dielectric material forming a tubular shape; and
depositing an outer conductor on an outer surface of the layer of dielectric material.

24. The method of claim 23, wherein depositing an outer conductor comprises:
depositing a layer of a conductive material on the layer of dielectric material; and
removing one or more portions of the layer of conductive material such that a strip of the conductive material is left on the dielectric material, the strip of the conductive material having a predetermined pattern.

25. The method of claim 24, wherein the predetermined pattern is one of a helical, fractal, or pseudo-fractal pattern.

26. The method of claim 23, wherein providing an inner conductor comprises:
providing a support rod;
depositing a layer of a conductive material on the support rod; and
removing one or more portions of the layer of conductive material such that a strip of the conductive material is left on the support rod, the strip of the conductive material having a predetermined pattern.

27. The method of claim 23, further comprising:
connecting the inner conductor to one of a radio frequency source or ground; and
connecting the outer conductor to the other of the radio frequency source or the ground.

28. A microwave ablation (MWA) device comprising:
a probe member; and
a microstrip antenna element disposed within the probe member, the microstrip antenna element comprising:
a dielectric substrate having a dielectric constant of between about 4 and about 30, the having a first substantially flat surface and a second substantially flat surface, the second surface being opposite the first surface;
a first conductor disposed on the first surface of the dielectric substrate; and
a second conductor disposed on a second surface of the dielectric substrate, the second conductor being a microstrip trace; and
wherein the first conductor is configured to be electrically connected to one of a radio frequency source or ground, and the second conductor is configured to be electrically connected to the other of the radio frequency source or the ground.

29. The MWA antenna of claim 28, wherein one or more of the first conductor and second conductor is connected to a plurality of conductive particles.

30. A radio frequency ablation (RFA) device comprising:
a RFA ablation probe member; and
a helical dipole antenna element disposed within the probe member, the helical dipole antenna element comprising:
a first conductor; and
a second conductor, wherein each of the first conductor and the second conductor extend in a substantially parallel direction along a longitudinal axis of the helical dipole antenna to a center point of the helical dipole antenna, the first conductor being wound helically about the longitudinal axis in a distal direction from the center point, and the second conductor being wound helically about the longitudinal axis in a proximal direction from the center point.
